(19) United States
(12) Patent Application Publication
(10) Pub. No.: US 2022/0163471 A1
(43) Pub. Date: May 26, 2022

(54) MOISTURE OR SATURATION ESTIMATION OF ABSORBENT ARTICLE

(71) Applicant: Raja Singh Tuli, Montreal (CA)

(72) Inventor: Raja Singh Tuli, Montreal (CA)

(21) Appl. No.: 17/519,071

(22) Filed: Nov. 4, 2021

Related U.S. Application Data
(60) Provisional application No. 63/117,555, filed on Nov. 24, 2020.

(51) Int. Cl. G01N 27/04; A61F 13/42

(52) U.S. Cl. CPC G01N 27/048; A61F 2013/424; A61F 13/42

(57) ABSTRACT

A method is provided for estimating the moisture or saturation level of an absorbent article, comprising: applying to a drive electrode embedded in the absorbent article a periodic drive signal; detecting a sense signal from a sense electrode embedded in the absorbent article, wherein the sense signal is a periodic pulse signal caused by the drive signal when there is moisture in the absorbent article; calculating variation rate or variation of the monotonically gradually varying envelop of the detected sense signal for a predetermined number of the periods of the periodic drive signal; and estimating the moisture level of the absorbent article based on the calculated variation rate or variation.

### CROSS-REFERENCE TO RELATED APPLICATIONS

This application claims priority to U.S. provisional patent application No. 63/117,555, entitled “MOISTURE OR SSTURATION ESTIMATION OF ABSORBENT ARTICLE,” filed on Nov. 24, 2020. The content of this U.S. provisional patent application is hereby incorporated by reference in its entirety for all purposes.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not Applicable

### STATEMENT REGARDING PRIOR DISCLOSURES BY THE INVENTOR OR A JOINT INVENTOR

Not Applicable

### BACKGROUND OF THE INVENTION

### Field of the Invention

The present disclosure generally relates to absorbent article, and specifically to moisture or saturation estimation of absorbent article.

### Description of Related Art

Disposable absorbent article such as disposable diaper is a product that is capable of receiving and retaining bodily exudates or excretions so as to prevent contamination of the clothing or external environment. As an example, with a disposable diaper, the user is allowed to urinate or defecate without the use of a toilet. In addition to diapers, there are numerous other types of disposable absorbent articles such as e.g. under pads, incontinence pads, fitted briefs, belted shields, liners, all-in-one pads, pull-up incontinence pants, training pants, protective underwear, catamenial napkins, and incontinence guards etc. It is to be understood that the list of disposable absorbent articles identified above is not exhaustive and that these and other absorbent articles can be used with the present disclosure and are within the scope of the present disclosure. It is also to be understood that a reference in this specification to any one such article, such as a “diaper” is to be taken to be a reference to any and all other suitable absorbent articles including incontinence garments, pads and the like.

In order to prevent contamination of the clothing or external environment, disposable absorbent article is provided with an absorbent core (e.g. absorbent pad) capable of receiving and retaining bodily exudates or excretions, and a substantially liquid impervious layer. In general, disposable absorbent products consist of a layered construction, which allows the bodily exudates or excretions to be distributed and transferred to the absorbent core where they are retained in. In everyday use, a disposable absorbent article may be used until the absorbent core is saturated with e.g. bodily exudates or excretions. When the absorbent core is saturated, the disposable absorbent article needs to be removed, disposed of, and replaced with a clean and dry article.

For many years, a variety of designs have been developed for detecting the moisture and/or saturation of the absorbent articles. However, most of these designs, e.g. as disclosed in U.S. application Ser. Nos. 15/818,136, 15/890,901, and 16/835,543, rely in general on the change of resistance, conductivity and/or impedance, which might results in false and/or inaccurate signals e.g. when the sensors remain in contact with the absorbent layer.

Thus, it is desirable to provide moisture and/or saturation estimation of absorbent article based on other parameters and/or characteristics in/of the absorbent article, to complement or replace the existing method.

### BRIEF SUMMARY OF THE INVENTION

Embodiments are presented herein of, inter alia, moisture or saturation estimation of absorbent article.

In an embodiment of the present disclosure, a method is provided for estimating the moisture or saturation level of an absorbent article, comprising: applying to a drive electrode embedded in the absorbent article a periodic drive signal; detecting a current from a sense electrode embedded in the absorbent article, wherein the current is a periodic pulse signal caused by the drive signal when there is moisture in the absorbent article; calculating reduction rate or reduction of the monotonically decreasing envelop of the detected current for a predetermined number of the periods of the periodic drive signal; and estimating the moisture level of the absorbent article based on the calculated reduction rate or reduction.

In a further embodiment of the present disclosure, a method is provided for estimating the moisture or saturation level of an absorbent article, comprising: applying to a drive electrode embedded in the absorbent article a periodic drive signal; detecting a sense signal from a sense electrode embedded in the absorbent article, wherein the sense signal is a periodic pulse signal caused by the drive signal when there is moisture in the absorbent article; calculating variation rate or variation of the monotonically gradually varying envelop of the detected sense signal for a predetermined number of the periods of the periodic drive signal; and estimating the moisture level of the absorbent article based on the calculated variation rate or variation.

In another further embodiment of the present disclosure, a method is provided for estimating the moisture or saturation level of an absorbent article, comprising: applying to a drive electrode embedded in the absorbent article a drive signal; detecting a current from a sense electrode embedded in the absorbent article; calculating a reduction of the decreased amount of the detected current; and estimating the moisture level of the absorbent article based on the calculated reduction.

This summary is intended to provide a brief overview of some of the subject matter described in this document. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE DRAWINGS

The various preferred embodiments of the present invention described herein can be better understood by those skilled in the art when the following detailed description is read with reference to the accompanying drawings. The components in the figures are not necessarily drawn to scale and any reference numeral identifying an element in one drawing will represent the same element throughout the drawings. The figures of the drawing are briefly described as follows.

FIG. 1 is a sectional view of a system for diaper moisture detection, according to an embodiment of the present disclosure.

FIG. 2 illustrates an example drive signal Sd versus time t graph and an example corresponding sense signal Ss versus time t graph, according to an embodiment of the present disclosure.

FIG. 3 illustrates another example drive signal Sd versus time t graph and an example corresponding sense signal Ss versus time t graph, according to an embodiment of the present disclosure.

FIG. 4 illustrates a further example drive signal Sd versus time t graph and an example corresponding sense signal Ss versus time t graph, according to an embodiment of the present disclosure.

While the features described herein are susceptible to various modifications and alternative forms, specific embodiments thereof are shown by way of example in the drawings and are herein described in detail. It should be understood, however, that the drawings and detailed description thereto are not intended to be limiting to the particular form disclosed, but on the contrary, the intention is to cover all modifications, equivalents and alternatives falling within the spirit and scope of the subject matter as defined by the appended claims.

### DETAILED DESCRIPTION OF THE INVENTION

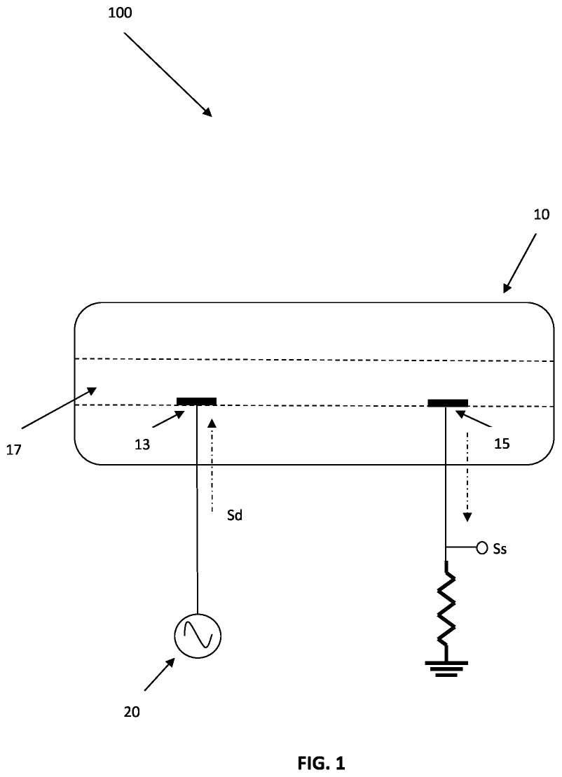

FIG. 1 is a sectional view of an example system 100 for diaper moisture detection according to an embodiment of the present disclosure. The system 100 for diaper moisture detection comprises a diaper 10, in which two electrodes, a drive electrode 13 and a sense electrode 15, are embedded e.g. directly above or directly underneath or even in the absorbent pad 17 of the diaper 10. These two electrodes 13 and 15 are spaced apart from each other such that when the diaper 10, in particular the absorbent pad 17, contains no moisture there exists no electrical path between these two electrodes and such that moisture, e.g. resulted from the user's urination or defecation, contained in the absorbent pad 17 between these two electrodes will cause an electrical path to be established between them. As an example, the two electrodes 13 and 15 are two elongate electrodes that run in parallel with each other and along the longitude direction of the diaper 10 in an embodiment of the present disclosure. Preferably, the two elongate electrodes both run across the whole length of the diaper 10.

During the operation, a drive signal Sd is applied to the drive electrode 13. For example, in the embodiment as illustrated in FIG. 1, a signal generator 20 is electrically coupled to the drive electrode 13 and generates and applies a drive signal Sd to the drive electrode 13. When an electrical path exists between the drive and sense electrode 13 and 15, the drive signal Sd applied to the drive electrode 13 causes a sense signal Ss to be sensed from the sense electrode 15. As a non-limiting example, a resistor that connects the drive electrode 13 to ground may be used to sense the sense signal Ss, e.g. as illustrated in the embodiment as illustrated in FIG. 1.

As described above, when there is no moisture contained in the absorbent pad 17 of the diaper 10, no electrical path exists between the drive electrode 13 and the sense electrode 15, and therefore no sense signal will be sensed from the sense electrode 15 even when a drive signal Sd is applied to the drive electrode 13. On the other hand, when there exists moisture in the absorbent pad 17, e.g. when the user wearing the diaper 10 urinates or defecates in the diaper, an electrical path is formed between the drive electrode 13 and the sense electrode 15, and consequently a sense signal Ss will be sensed from the sense electrode 15 when a drive signal Sd is applied to the drive electrode 13.

As an example, the signal generator 20 is electrically coupled to the drive electrode 13 via direct contact, and generates and applies a square wave voltage as the drive signal Sd to the drive electrode 13 in an embodiment of the present disclosure. As will be understood by those skilled in the art, a square wave signal is a periodic signal with a period of T and a duty cycle of D. For the present disclosure, the period T of the square wave voltage Sd may be set as m seconds or millisecond where m can be selected from a value range from 1 to 5 as an example. The duty cycle D is represented by a percentage ranging from 0% to 100% non-inclusive.

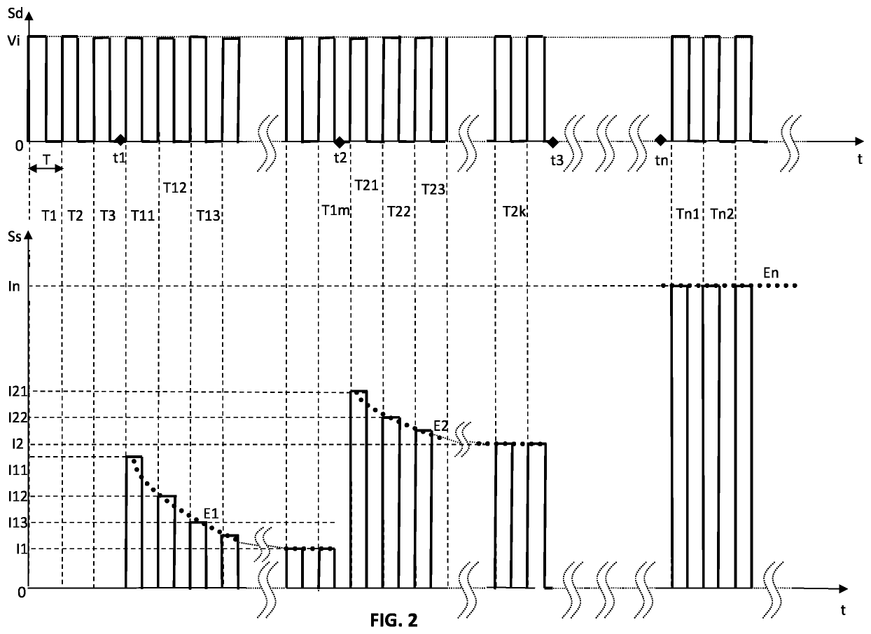

Consider, as an example, an embodiment where the signal generator 20 is electrically coupled to the drive electrode 13 via direct contact, and generates and applies a square wave voltage as the drive signal Sd to the drive electrode 13. An example drive signal Sd versus time t graph and an example corresponding sense signal Ss versus time t graph are illustrated in FIG. 2.

In FIG. 2, the upper graph represents the drive signal Sd (which is a square wave voltage for this specific embodiment as an example) versus time t graph. It can be seen from this upper graph that, in this specific embodiment the duty cycle D is set as 50%, and the drive signal Sd represents a voltage of Vi for the first half of one period while represents 0 or a tri-state (i.e. disconnected, or high impedance state) for the second half of the one period.

Let us consider the case where the user urinates in the diaper 10 at t1 for the first time. That is, until t1 no moisture is contained in the diaper 10, in particular in the absorbent pad 17. Therefore, until t1 there is no sense signal Ss being sensed from the sense electrode 15, even when the voltage Vi is applied to the drive electrode 13 during the first halves of the periods T1, T2 and T3 respectively.

For the first period T11 of the square wave signal Sd after t1, a voltage Vi is applied again to the drive electrode 13 during T11's first half. At this time, there already exists moisture in the diaper 10, in particular in the absorbent pad 17 that is resulted from the user's urination at t1. The moisture in the absorbent pad 17 establishes an electrical path between the drive electrode 13 and the sense electrode 15, which results in a current I11 (i.e. current lo, as the sense signal Ss) being sensed from the sense electrode 15 during the first half of this period T11.

An example sense signal Ss (i.e. current lo) versus time t graph is illustrated in the lower graph of FIG. 2. As illustrated, no current is sensed from the sense electrode 15 during the first three periods T1, T2 and T3 because no electrical path exists between the drive electrode 13 and the sense electrode 15. After the user urinates in the diaper 10 at t1 for the first time, an electrical path is established between the drive and sense electrode 13 and 15. Therefore, a current I11 is sensed from the sense electrode 15 during the first half of the period T11. Next, during the second half of T11, no current is sensed from the sense electrode 15 because the voltage Vi is removed from the drive electrode 13.

For the next period T12, the voltage Vi is applied to the drive electrode 13 again during its first half, and thus a current I12 is sensed from the sense electrode 15 during this half period. It is to be noted that the amplitude of this current I12 is reduced when compared to the current I11. This reduction of current is resulted from the increase in charges that are absorbed in the absorbent pad 17, in particular in its particles (i.e. fibers and SAPs).

It will be understood by those skilled in the art that, the absorbent pad of a diaper is composed primarily of two elements: pulp, a fibrous material from trees; and superabsorbent polymer (SAP) that is made of fine dots or spheres of plastics and can absorb liquid very much.

Like in a flash memory, the fibers and SAPs in the absorbent pad absorb charges when a voltage is applied, just like nano particles, as long as the fibers and SAPs are not super wet, e.g. as long as the diaper is not 100% saturated. And the charges as absorbed will remain there for e.g. minutes, hours, etc.

Referring back to FIG. 2, during the first half of T1, a voltage Vi is applied to the drive electrode 13, resulting in some charges being absorbed and stored in the fibers and SAPs. With a voltage Vi being applied again during the first halves of T2 and T3 respectively, more and more charges are absorbed and stored in the fibers and SAPs. However, when the user urinates in the diaper 10 for the first time at t1, the charges absorbed in the absorbent pad 17 dissipate.

After t1, a voltage Vi is applied again to the drive electrode 13 during the first half of the first period T11 after t1, resulting in a current I11 e.g. flowing from the drive electrode 13 to the sense electrode 15 and also resulting in some charges being absorbed and stored in the fibers and SAPs.

It will be appreciated by those skilled in the art that those charges absorbed and stored in the fibers and SAPs may be opposing the current flow from the drive electrode 13 to the sense electrode 15. Therefore, when a voltage Vi is applied again to the drive electrode 13 during the first half of the next period T12, more charges are absorbed and stored in the fibers and SAPs, that is, now in the absorbent pad 17 there exist more charges e.g. opposing the current flow from the drive electrode 13 to the sense electrode 15, which in turn results in the decrease in current. Consequently, the current I12 during the first half of the period T12 is reduced when compared to the current I11 during the first half of the period T11.

Similarly, the currents I13, I14, . . . for the next periods T13, T14, . . . further decrease gradually. Thus, as illustrated in FIG. 2, the envelope E1 of the sense signal Ss (i.e. the current Io) after the first urination at t1 decreases gradually with time, and will finally become substantially constant at a value I1 when the charges in the absorbent pad 17 between the two electrodes 13 and 15 become saturated. It is to be noted that, the falling portion in the envelope E1 of the sense signal Ss (such as its rate of decrease, etc.) can be used to determine the properties related to the moisture of the diaper 10 after the first urination at t1, such as the moisture level and the saturation level of the diaper 10.

Now, let us consider the case where the user wearing the diaper 10 makes another urination at t2. It is to be understood that, the second urination at t2 causes the diaper 10 to contain more moisture and also causes the charges already stored in the particles in the absorbent pad to dissipate.

For the first period T21 after t2, a current I21 is sensed from the sense electrode 15 when a voltage Vi is applied to the drive electrode 13 during the T21's first half. Since the second urination at t2 makes the diaper 10 contain more moisture, the diaper 10 has a lower resistance between the two electrodes 13 and 15. Therefore, the current I21 is greater than the current I11.

Again, the decrease in current occurs for the next periods T22, T23, T24, . . . in the same way as that is described above with respect to the periods T12, T13, T14, . . . . That is, starting from the period T21, the envelope E2 of the sense signal Ss (i.e. the current Io) also decreases gradually with time, and will finally become substantially constant at a value I2 when the charges in the absorbent pad 17 between the two electrodes 13 and 15 become saturated. Similarly, this falling portion in the envelope E2 of the sense signal Ss can be used to determine the properties related to the moisture of the diaper 10 after the second urination at t2.

It is to be noted that, compared with the envelope E1 after the first urination at t1, the rate of decrease for the falling portion is reduced in the envelope E2 of the sense signal Sd after the second urination at t2. That is because, when the absorbent pad 17 becomes moister (i.e. contains more moisture), the fibers and SAPs in the absorbent pad 17 become less able to carry charge, or there exist less particles (fibers and SAPs) in the absorbent pad 17 that can carry the charge. As a result, compared with the periods between t1 and t2, the decrease in current is less during the periods after t2, which results in the rate of decrease for the falling portion being reduced for the envelope E2 when compared with the envelope E1.

The same process repeats for the envelope(s) after the next urination(s) at t3, . . . , but with the rate of decrease for the falling portion being further reduced gradually.

Finally, the diaper 10 becomes 100% saturated after the user's nth urination at tn (where n is an integer greater than or equal to e.g. 4), which results in the lowest resistance of the diaper 10 (in particular, between the two electrodes 13 and 15) and also causes the charges previously stored in the particles in the absorbent pad 17 to dissipate.

With the lowest resistance of the diaper 10, the highest current In is sensed from the sense electrode 15 when a voltage Vi is applied to the drive electrode 13 during the first half of the first period Tn1 after tn. As will be understood by those skilled in the art that when the diaper 10 is 100% saturated, there exists no particle (fiber and SAP) in the absorbent pad 17 that can carry charge, therefore, no charge will be absorbed and stored in the absorbent pad 17. Consequently, there is no decrease in current for the next periods, that is, the current In will remain unchanged for the next periods, in particular during their first halves, as illustrated in FIG. 2. That is, there is no decrease (i.e. the rate of decrease is zero) in the envelope En of the sense signal Ss (i.e. the current Io) after the nth urination at tn.

It is to be noted that, based on the variation in the rate of decrease for the falling portions of the envelopes of the sense signal Ss (i.e. the current Io), the properties related to the moisture of the diaper 10 such as the moisture level and the saturation level can be determined.

As will be understood by those skilled in the art, for applying the drive signal Sd to the drive electrode 13 and for sensing the sense signal Ss from the sense electrode 15, it is much more convenient to establish an electrical coupling via capacitive coupling contact than via direct contact. And sometimes it is very difficult or even impossible to make an electrical coupling via direct contact in which case a capacitive coupling contact is preferred or necessary for the electrical connection.

It is to be noted that the above-described example process as illustrated in FIG. 2 does not apply for the capacitive coupling, because for the capacitive coupling, the sense signal Ss (i.e. current Io) only occurs when there exists change in the drive signal Sd. In particular, when the drive signal Sd is a square wave voltage, the sense signal Ss only occurs when the drive signal Sd steps up or falls down, i.e. at the rising edge and the falling edge of the drive signal Sd. When the drive signal Sd steps up, the sense signal Ss (i.e. current Io) occurs in one direction, e.g. from the drive electrode 13 to the sense electrode 15, and some charges appear in the diaper 10. On the other hand, when the drive signal Sd falls down, the sense signal Ss (i.e. current Io) with the same amplitude occurs in the opposite direction, e.g. from the sense electrode 15 to the sense electrode 13, which in turn removes all the charges in the diaper 10. Therefore, the above-described process will not be observed in the sense signal Ss, and we cannot determine the properties related to the moisture of the diaper based on such process.

With capacitive coupling, when a square wave drive signal is driven into the drive electrode, a sense signal is sensed from the sense electrode in which a signal spike goes up at the rising edge of the drive signal and a signal spike goes down at the falling edge of the drive signal. In an embodiment of the present disclosure, only the spikes going down at the falling edge of the drive signal are examined.

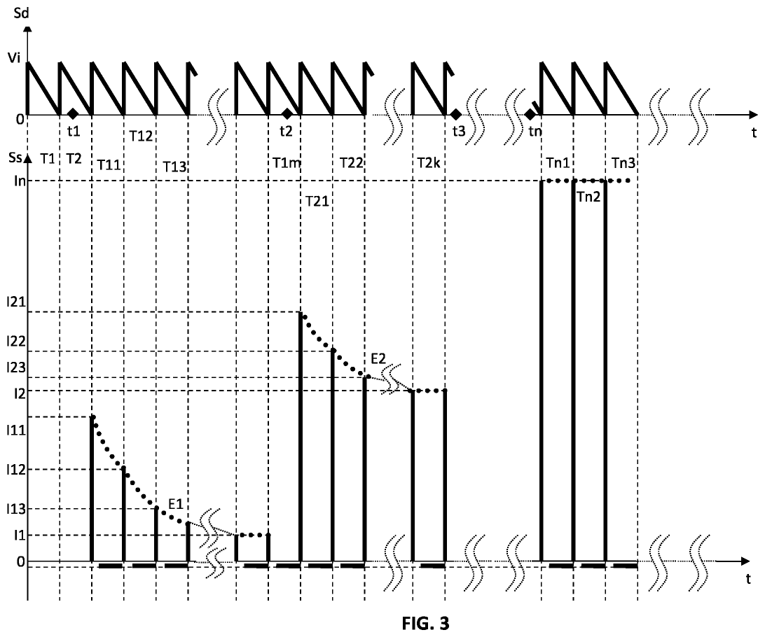

When the electrical coupling is performed via capacitive coupling contact, a sawtooth wave signal (voltage) as illustrated in the upper graph of FIG. 3 can be used as the drive signal Sd, as an example in an embodiment of the present disclosure. As can be seen from the upper graph in FIG. 3, the drive signal Sd is a periodic signal with a period T. For the period from the time IT to the time (I+1)T, the drive signal Sd steps up to Vi at the time IT, and then declines linearly to zero during the rest of this period, where I is an integer greater than or equal to 0.

With this sawtooth wave voltage Sd being applied to the drive electrode 13, if an electrical path exists between the drive electrode 13 and the sense electrode 15, a pulse current flows e.g. from the drive electrode 13 to the sense electrode 15 when the sawtooth wave voltage Sd steps up from zero to Vi, which also result in some charges being absorbed and stored in the diaper, in particular between the two electrodes 13 and 15. Next, when the sawtooth wave voltage Sd declines from Vi to zero linearly, a very small current flows e.g. from the sense electrode 15 to the drive electrode 13. Since the linear decline of the drive signal Sd is relative gentle while the rising edge of the drive signal Sd from zero to Vi is extremely steep, this current flow from the sense electrode 15 to the drive electrode 13 is small enough to be ignored when compared to the pulse current flow from the drive electrode 13 to the sense electrode 15. This current flow from the sense electrode 15 to the drive electrode 13 also removes some charges from the diaper. However, because of its very small amplitude, this current flowing from the sense electrode 15 to the drive electrode 13 only removes a very small amount of charges from the diaper.

An exemplary sense signal Ss (i.e. current Io) versus time t graph is illustrated in the lower graph of FIG. 3 with respect to the exemplary sawtooth drive signal Sd in the upper graph of FIG. 3.

As illustrated, for the first two periods T1 and T2, i.e. from the time 0 to the time 2T, there is no current Io (i.e. the sense signal Ss) being sensed from the sense electrode 15 because there exists no electrical path between the drive electrode 13 and the sense electrode 15.

Then, the user wearing the diaper 10 urinates or defecates in the diaper at t1 for the first time, which results in an electrical path being established between the drive electrode 13 and the sense electrode 15 and also results in the dissipation of the charges (if any) in the diaper.

For the first period T11 after t1, a pulse current I11 flows e.g. from the drive electrode 13 to the sense electrode 15 when the drive signal Sd steps up from zero to Vi at the beginning of this period T11, i.e. at the rising edge of the drive signal Sd. This rising edge of the drive signal Sd also results in some charges being absorbed and stored in the diaper.

Next, the drive signal Sd declines linearly from Vi to zero for the rest of this period, which results in a very small current I11′ e.g. flowing from the sense electrode 15 to the drive electrode 13. And most of the charges that are absorbed and stored in the diaper at the rising edge of the drive signal Sd remain during the linear decline of the drive signal Sd, because the very small current I11′ during the linear decline removes only a very small amount of charges from the diaper.

Then for the next period T12, when the drive signal Sd steps up from zero to Vi, more charges are absorbed and stored in the diaper, and a pulse current I12 flows from the drive electrode 13 to the sense electrode 15. Since in the diaper more charges are absorbed that are opposing the current flow from the drive electrode 13 to the sense electrode 15, the current I12 is reduced when compared to the current I11.

For the rest of this period T12, as the drive signal Sd declines linearly from Vi to zero, a very small current I12′ flows from the sense electrode 15 to the sense electrode 13, which in turn removes a very small amount of charges from the diaper.

Similarly, the decrease in current flow from the drive electrode 13 to the sense electrode 15 repeats for the next periods. Consequently, the envelope E1 of this current flow from the drive electrode 13 to the sense electrode 15 for the periods after the first urination at t1 decrease with time, and becomes substantially constant at a value I1 when the charges in the absorbent pad 17 between the two electrodes 13 and 15 become saturated. It is to be noted that the falling portion of the envelope E1 can be used to determine the properties related to the moisture of the diaper 10 after the first urination at t1.

Similarly to the process as described above with respect to FIG. 2, with additional urinations in the diaper 10, the pulse currents flowing e.g. from the drive electrode 13 to the sense electrode 15 become higher and higher, and the rates of decrease of the falling portions in their envelopes are reduced gradually. And finally, when the diaper 10 becomes 100% saturated, the pulse current flowing from the drive electrode 13 to the sense electrode 15 becomes the highest current, and the rate of decrease of the falling portion in its envelope becomes zero, i.e. there is no more decrease in current. All these characteristics can be used to determine the properties related to the moisture of the diaper 10 after urination(s).

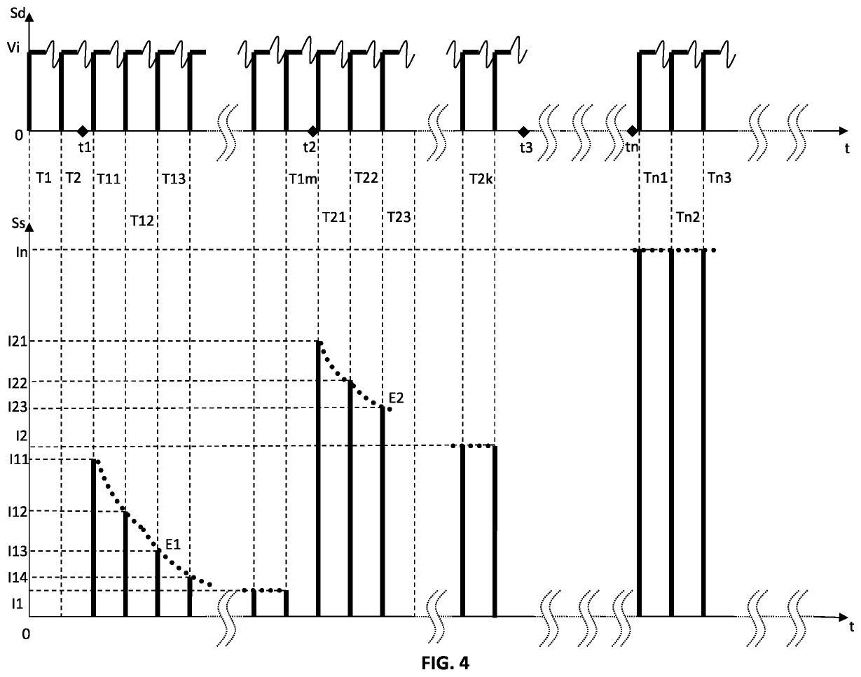

In another alternative embodiment of the present disclosure, the electrical coupling is also performed via capacitive coupling contact, and a signal (voltage) as illustrated in the upper graph of FIG. 4 is used as the drive signal Sd as an example.

In the embodiment as illustrated in FIG. 4, this drive signal Sd has a period T and a duty cycle D of 50%, similarly to the square wave signal as illustrated in FIG. 2. It can be seen from the upper graph in FIG. 4 that, the drive signal Sd steps up to Vi at the beginning of each period, and keeps Vi for the first half of the period T. Next, the drive signal Sd becomes tristate (i.e. disconnected or high impedance state) for the second half of the period T. In this way, there exist two transitions in this drive signal Sd during one period, i.e. stepping up to Vi and changing from Vi to tristate.

With the drive signal Sd stepping up to Vi, some charges are absorbed in the diaper, and a pulse current flows e.g. from the drive electrode 13 to the sense electrode 15 if there exists an electrical path between these two electrodes. On the other hand, when the drive signal Sd changes from Vi to tristate, because of the tristate, i.e. the disconnected or high impedance state, no current flows between the drive electrode 13 and the sense electrode 15, which results in no charge being removed from the diaper at this transition in the drive signal Sd.

An exemplary sense signal Ss (i.e. current Io) versus time t graph is illustrated in the lower graph of FIG. 4 with respect to the exemplary drive signal Sd in the upper graph of FIG. 4.

As illustrated, for the first two periods T1 and T2, i.e. from the time 0 to the time 2T, there is no current Io (i.e. the sense signal Ss) being sensed from the sense electrode 15 because there exists no electrical path between the drive electrode 13 and the sense electrode 15.

Then, the user wearing the diaper 10 urinates or defecates in the diaper at t1 for the first time, which results in an electrical path being established between the drive electrode 13 and the sense electrode 15 and also results in the dissipation of the charges (if any) in the diaper.

For the first period T11 after t1, a pulse current I11 flows e.g. from the drive electrode 13 to the sense electrode 15 when the drive signal Sd steps up to Vi at the beginning of this period T11, i.e. at the rising edge of the drive signal Sd. This rising edge of the drive signal Sd also results in some charges being absorbed and stored in the diaper.

The drive signal Sd keeps Vi for the first half period, which however produces no current because there is no transition in the drive signal Sd. Next, the drive signal Sd changes from Vi to tristate, which also produces no current because of the tristate. Similarly, when the drive signal Sd keeps tristate during the second half of the period, no current is produced because no transition occurs.

Then for the next period T12, when the drive signal Sd steps up to Vi, more charges are absorbed and stored in the diaper, and a pulse current I12 flows e.g. from the drive electrode 13 to the sense electrode 15. Since in the diaper more charges are absorbed that are opposing the current flow from the drive electrode 13 to the sense electrode 15, the current I12 is reduced when compared to the current I11. For the rest of this period T12, no current occurs, just like in the preceding period.

Similarly, the decrease in pulse current from the drive electrode 13 to the sense electrode 15 repeats for the next periods. Consequently, the envelope E1 of this pulse current e.g. from the drive electrode 13 to the sense electrode 15 for the periods after the first urination at t1 decrease with time, and becomes substantially constant at a value I1 when the charges in the absorbent pad 17 between the two electrodes 13 and 15 become saturated. It is to be noted that the falling portion of the envelope E1 can be used to determine the properties related to the moisture of the diaper 10 after the first urination at t1.

Similarly to the process as described above with respect to FIG. 2, with additional urinations in the diaper 10, the pulse currents e.g. from the drive electrode 13 to the sense electrode 15 become higher and higher, and the rates of decrease of the falling portions in their envelopes are reduced. And finally, when the diaper 10 becomes 100% saturated, the pulse current e.g. from the drive electrode 13 to the sense electrode 15 become the highest current, and the rate of decrease of the falling portion in its envelope becomes zero, i.e. there is no more decrease in current. All these characteristics can be used to determine the properties related to the moisture of the diaper 10 after urination(s).

It is to be noted that the above-described embodiments of the present disclosure are not time-dependent. Instead, the sense signal behaves depending on the amount of the pulses received from the drive signal, in particular how many charges are injected in the drive signal. In other words, the envelope of the sense signal after one urination or defecation depends on the amount of pulses (e.g. the square pulses) received from the drive signal, instead of on the amount of time. Therefore, in order to determine the behaviour of the envelope of the sense signal, it is the decrease of the envelope after a certain number of pulses (instead of after a certain time) to be checked. It is understood that a certain number of pulses e.g. 10 pulses could correspond to different times, e.g. 10 second or 10 ms. In the embodiments of the present disclosure, based on how much decrease of the envelope after a certain number of pulses, it could be determined e.g. the charge carrying capability of the absorbent material, the wetness and the saturation of the diaper.

Please also note that all the above-described embodiments in the present disclosure can apply to measure saturation. It will be understood that the scope of the present disclosure is intended to encompass not only the drive electrode and the sense electrode in the form of two straight lines, but also the drive electrode and the sense electrode in any appropriate form, like multiple sections.

It is to be noted that although the present disclosure has been described with respect to diaper, the present disclosure is not limited to diaper. Instead, it is intended that the present disclosure encompass any absorbent article.

Although the embodiments above have been described in considerable detail, numerous variations and modifications will become apparent to those skilled in the art once the above disclosure is fully appreciated. It is intended that the following claims be interpreted to embrace all such variations and modifications.

The invention claimed is:

1. A method for estimating the moisture or saturation level of an absorbent article, comprising:
applying to a drive electrode embedded in the absorbent article a periodic drive signal;
detecting a current from a sense electrode embedded in the absorbent article, wherein the current is a periodic pulse signal caused by the drive signal when there is moisture in the absorbent article;
calculating reduction rate or reduction of the monotonically decreasing envelop of the detected current for a predetermined number of the periods of the periodic drive signal; and
estimating the moisture level of the absorbent article based on the calculated reduction rate or reduction.

2. The method of claim 1, wherein when moisture in the absorbent article remains constant, the amplitude of the current decreases monotonically gradually due to the charges absorbed in the absorbent article caused by the drive signal, until the charges absorbed in the absorbent article reach saturation.

3. The method of claim 2, wherein when a wetness event occurs in the absorbent article, the charges absorbed in the absorbent article dissipate and the amplitude of the current increase abruptly.

4. The method of claim 2, wherein the charges are absorbed in the absorbent article over the periods of the drive signal, until the charges absorbed in the absorbent article reach saturation.

5. The method of claim 2, wherein when there is more moisture in the absorbent article, less charges are absorbed in the absorbent article and thus the reduction rate or reduction is lower.

6. The method of claim 2, wherein when moisture in the absorbent article remains constant, the amplitude of the current remains constant after the charges absorbed in the absorbent article reach saturation.

7. The method of claim 1, wherein the drive signal is a square wave signal and wherein both the application of the drive signal and the detection of the current are performed with direct contact.

8. The method of claim 1, wherein the drive signal is a sawtooth wave signal, or a period signal with two states one of which is tristate, and wherein the application of the drive signal and/or the detection of the current are performed with capacitive coupling.

9. The method of claim 1, wherein the drive electrode and the sense electrode are arranged in parallel and run in the longitudinal direction of the absorbent article.

10. A method for estimating the moisture or saturation level of an absorbent article, comprising:
applying to a drive electrode embedded in the absorbent article a periodic drive signal;
detecting a sense signal from a sense electrode embedded in the absorbent article, wherein the sense signal is a periodic pulse signal caused by the drive signal when there is moisture in the absorbent article;
calculating variation rate or variation of the monotonically gradually varying envelop of the detected sense signal for a predetermined number of the periods of the periodic drive signal; and
estimating the moisture level of the absorbent article based on the calculated variation rate or variation.

11. The method of claim 10, wherein the amplitude of the sense signal increases when there is more moisture in the absorbent article.

12. The method of claim 10, wherein when moisture in the absorbent article remains constant, the amplitude of the sense signal varies monotonically gradually due to the charges absorbed in the absorbent article caused by the drive signal, until the charges absorbed in the absorbent article reach saturation.

13. The method of claim 12, wherein the charges are absorbed in the absorbent article over the periods of the drive signal, until the charges absorbed in the absorbent article reach saturation.

14. The method of claim 12, wherein when there is more moisture in the absorbent article, less charges are absorbed in the absorbent article and thus the variation rate or variation is lower.

15. The method of claim 12, wherein when moisture in the absorbent article remains constant, the amplitude of the sense signal remains constant after the charges absorbed in the absorbent article reach saturation.

16. The method of claim 12, wherein when a wetness event occurs in the absorbent article, the charges absorbed in the absorbent article dissipate and the amplitude of the sense signal increase abruptly.

17. The method of claim 10, wherein the drive signal is a square wave signal and wherein both the application of the drive signal and the detection of the sense signal are performed with direct contact.

18. The method of claim 10, wherein the drive signal is a sawtooth wave signal, or a period signal with two states one of which is tristate, and wherein the application of the drive signal and/or the detection of the sense signal are performed with capacitive coupling.

19. The method of claim 10, wherein the drive electrode and the sense electrode are arranged in parallel and run in the longitudinal direction of the absorbent article.

20. A method for estimating the moisture or saturation level of an absorbent article, comprising:
applying to a drive electrode embedded in the absorbent article a drive signal;
detecting a current from a sense electrode embedded in the absorbent article;
calculating a reduction of the decreased amount of the detected current; and
estimating the moisture level of the absorbent article based on the calculated reduction.

21. The method of claim 20, wherein when moisture in the absorbent article remains constant, the amplitude of the current decreases monotonically gradually due to the charges absorbed in the absorbent article caused by the drive signal, until the charges absorbed in the absorbent article reach saturation.

22. The method of claim 21, wherein when a wetness event occurs in the absorbent article, the charges absorbed in the absorbent article dissipate and the amplitude of the current increase abruptly.

23. The method of claim 21, wherein the charges are absorbed in the absorbent article, until the charges absorbed in the absorbent article reach saturation.

24. The method of claim 21, wherein when there is more moisture in the absorbent article, less charges are absorbed in the absorbent article and thus the reduction is lower.

25. The method of claim 21, wherein when moisture in the absorbent article remains constant, the amplitude of the current remains constant after the charges absorbed in the absorbent article reach saturation.

26. The method of claim 20, wherein the drive signal is a square wave signal and wherein both the application of the drive signal and the detection of the current are performed with direct contact.

27. The method of claim 20, wherein the drive signal is a sawtooth wave signal, or a period signal with two states one of which is tristate, and wherein the application of the drive signal and/or the detection of the current are performed with capacitive coupling.

28. The method of claim 20, wherein the drive electrode and the sense electrode are arranged in parallel and run in the longitudinal direction of the absorbent article.
